(19) United States
(12) Patent Application Publication
(10) Pub. No.: US 2018/0147749 A1
(43) Pub. Date: May 31, 2018

(54) Forming Pocket And Method For Making A Forming Pocket

(71) Applicant: GDM S.p.A., Bologna (IT)

(72) Inventors: Matteo PIANTONI, Albino (Bergamo) (IT); Valerio SOLI, Bologna (IT)

(21) Appl. No.: 15/570,398

(22) PCT Filed: May 3, 2016
(86) PCT No.: PCT/IB2016/052515

§ 371 Date: Oct. 30, 2017

(30) Foreign Application Priority Data
May 4, 2015 (IT) BO2015A000222

(51) Int. Cl. B29C 33/38; A61F 13/15; B22F 5/00; B33Y 10/00; B33Y 80/00

(52) U.S. Cl. CPC B29C 33/3842; A61F 13/15626; A61F 13/15658; B22F 3/1055; B33Y 10/00; B33Y 80/00; B22F 5/007

(57) ABSTRACT

A pocket, to form particulate material as absorbent for hygienic products, includes: a substrate shaped to the absorbent padding; a grid-shaped support, couplable to support the substrate during suction of the particulate material through the substrate and includes a curved external face, an internal face, a pair of greater side faces and a pair of lesser side faces that are opposite one another, openings enable gas to flow from the external face to the internal face during suction. A method for making the pocket includes making at least one of the external openings of a different shape and/or dimension from one of the internal openings, and making the external walls and the internal walls by layer additive manufacturing.

### TECHNICAL FIELD

The present invention relates to a forming pocket for forming absorbent padding for hygienic products comprising an external forming substrate, suitable for receiving particulate material and forming conglomerates from the particulate material, and a grid-shaped supporting structure which is couplable with the external forming substrate. The present invention further relates to a method of making the grid-shaped supporting structure of the forming pocket by an additive manufacturing process.

The present invention is applied advantageously to a forming conveyor for forming hygienic products comprising a plurality of forming pockets that are suitable for forming respective absorbent conglomerates for hygienic products in a forming apparatus for forming hygienic products, to which reference will be made below without any loss of generality.

### BACKGROUND

As is known, hygienic products, in particular diapers for children, sanitary towels or products for adult incontinence comprise a layer of absorbent padding enclosed between a layer of nonwoven fabric and an impermeable layer, for example polyethylene. The absorbent padding is made of a conglomerate of cellulose fibres and/or particles of super-absorbent material which is formed in a forming apparatus for forming such hygienic products.

In order to make anatomically shaped hygienic products, it is known to shape the absorbent padding into the desired anatomical shape before enclosing the padding between the layer of nonwoven fabric and the impermeable layer.

The forming apparatus (which is not shown) comprises a forming drum 1 (shown schematically in FIGS. 1 to 3) of absorbent padding which is provided, on the external periphery thereof, with a plurality of sucking forming pockets, and is supplied, at the periphery, with a flow of particulate material. In each pocket, the fibres of the particulate material are conveyed by a sucking air flow and are compacted by suction, thus obtaining the absorbent conglomerate, known also as fluff, of the desired shape.

According to a different embodiment that is not shown, the forming conveyor can comprise a closed-loop continuous belt conveyor.

As shown in FIG. 1, the forming drum 1 comprises a plurality of forming pockets 2a that are shaped, aligned and uniformly distributed circumferally along the external surface of the drum 1, and comprising for example a cavity having substantially the shape of a truncated pyramid for making absorbent padding with variable thickness. Alternatively, as shown in FIG. 2b, the forming drum 1 can comprise forming pockets 2b that are shaped, aligned and uniformly distributed circumferally along the external surface of the drum 1, comprising for example an anatomical cavity of rounded shape for making absorbent padding of anatomical shape. Also alternatively, as shown in FIG. 3, the forming drum 1 can comprise a single forming pocket 2c that is shaped as a single annular cavity to make a web of absorbent padding, to be divided into rectangular portions with subsequent cutting processing.

Each forming pocket has in other words the right shape for the padding to be obtained and or to permit subsequent processing for which the absorbent conglomerate is intended. The depth of the forming pocket determines the thickness of the absorbent layer to be made. The power of the sucking air flow in a zone of the forming pocket determines the compactness and thus the density of the absorbent layer in that zone.

The forming pockets are typically fixed to compartments of the forming drum of a shape corresponding to the pockets.

The forming pockets have to be perforated to enable the air flow to retain effectively by suction the particles of which the particulate material consists on the surface but at the same time have to prevent the pulverised material, which also makes up the particulate material, from traversing the particles. The openings in the forming pockets thus have to be of reduced dimension and typically it is required for the openings to be of a dimension that is comprised in a range between 0.20 mm and 0.40 mm.

In order to make the forming pockets, usually of metal, it is known to use, to receive and retain the particulate material, micro-perforated metal foils or micro-perforated metal nets by means of which it is possible to make openings of the desired dimension. Nevertheless, such metal foils or such metal nets have a reduced thickness and are thus flexible and easily deformable.

The deformability of the forming pocket during assembling and/or disassembling of the pocket in the forming drum makes the handling and cleaning thereof difficult that is frequently prescribed by scheduled maintenance tasks to remove with deep cleaning possible particulate material lodged in the openings of the forming pocket.

In order to ensure suitable sturdiness for the forming pockets, to prevent possible deformation thereof and thus make assembling and/or disassembling of the forming pockets easier during the maintenance tasks, it is known to support the micro-perforated metal foil or the metal net, which make the external forming substrate, by means of a stiff support, which is also perforated to enable the air flow to pass through and is a support to the external substrate.

The external substrate is of a shape conjugated to the shape of the absorbent padding to be made whereas the stiff support is of a shape conjugated to the shape of the external substrate to support the external substrate appropriately and confer sturdiness thereupon.

As shown by U.S. Pat. No. 4,761,258 and illustrated in FIGS. 4 and 5 with reference to a forming pocket 2a shaped as in FIG. 1, the external forming substrate 3 is made of perforated metal foil, has a cavity 4 of substantially frustoconical shape and is supported by a supporting structure 5, made as a metal net, which has a corresponding cavity 6, shaped like the cavity 4 and arranged at the cavity 4 of the external forming substrate 3. Alternatively to the metal net 5, the supporting structure can be made by a metal grid 7, shown in FIG. 5 or by a honeycomb grid (not shown), which afford even greater sturdiness than the metal net and are thus usually preferred.

It should be noted that the absorbent padding that is obtainable from the forming pocket of FIG. 4 has a portion with a greater thickness at the frustoconical cavity 4 of the forming pocket 2a. Shaped padding with variable thickness obliges the external forming substrate 3 to have at least one zone that is concave like the cavity 4 and accordingly obliges also the external face of the supporting structure 5, intended to contact the external forming substrate 3, to have a respective concave zone, i.e. the cavity 6. It is added that when the forming pocket is fixed to the forming drum, the internal face of the supporting structure, opposite the external face, is also curved inasmuch as it is intended for contact with the forming drum 1.

WO 2008061178 also discloses a forming pocket of an apparatus for forming absorbent products made of fibrous material. The forming pocket comprises a plurality of different layers, i.e. a perforated forming surface made by means of a thin sheet micro-perforated by electro incision; a metal or titanium screen, which has a central opening; a central forming chamber which is superimposed on the central opening of the screen and houses internally a plurality of central wings and a peripheral forming chamber that surrounds the central forming chamber and houses internally a plurality of side wings.

Optionally, the forming pocket can comprise a grid-shaped central supporting structure, which is shaped and can be lodged in the central forming chamber resting on the central wings, and a grid-shaped edge supporting structure, which is also shaped, that can be lodged in the peripheral forming chamber resting on the plurality of side wings.

U.S. Pat. No. 6,098,249 discloses a modular pocket in a forming drum for forming absorbent material that is made by two end rails and two side rails to which are fixed a plurality of plates, by screws or other fixing means, which define the internal openings on the modular pocket.

US 2004/098838 discloses a forming pocket comprising a forming surface, transverse walls and longitudinal walls fixed to end walls, and to a honeycomb structure that is sustained by the transverse walls and by the longitudinal walls and is in turn a support to a perforated plate.

The grid supporting structures disclosed previously in relation to the prior-art documents, which are shaped with both a curved external face and with a curved internal face, are made from a flat grid, which is machined by a manufacturing method that provides a plurality of successive manufacturing steps to obtain a grid-shaped supporting structure provided with at least one hollow shaped zone.

After obtaining a flat grid by welding together a plurality of drawn sheets, the grid is first curved to obtain the internal face to be rested on the welding drum and is then treated by a spark discharge machining process to make the hollow zones of the external face.

One drawback is represented by the fact that the grid-shaped supporting structure has very high costs inasmuch as the productive process that is necessary for making the grid-shaped supporting structure is costly and requires significant industrial investments.

In fact, in order to obtain the supporting structure by the aforesaid method, first of all dedicated equipment has to be provided, like special welding benches and presses and specific spark discharge machining benches, which are very expensive. Further, the spark discharge machining process is in itself particularly costly as the electrodes thereof, which are subject to wear, require frequent regeneration. As several machining steps are further required, the method of producing each grid-shaped supporting structure is very long and require specialised workers.

It is added that the shape of each grid-shaped supporting structure is determined by the shape of the corresponding absorbent padding to be made and that thus the equipment dedicated to the production of a specific type of supporting structure is to be modified to the varying of the type of absorbent padding to be made.

A further problem of the supporting structure made by the previously illustrated method is linked to the fact that the compactness and thus the density of the absorbent layer in a zone of the forming pocket are not easily modifiable. As it is not possible to make a grid with differentiated sucking zones at moderate cost, the supporting structure cannot cooperate with the external substrate to determine the sucking air flow through the forming pocket. As a result, the density of the absorbent layer in a zone of the forming pocket is due exclusively to the position and to the diameter of the micro-openings present in the external substrate, which determine the sucking air flow and thus, given a determined sucking power, the lesser or greater retention on the forming sublayer of the particulate material. Nevertheless, the diameter of the micro-openings is mainly determined by the minimum diameter of the pulverised material that has to be retained and cannot thus be adapted to determine the desired density of the forming sublayer.

This greatly limits the possibility of differentiating the compactness of the absorbent layer inside the absorbent layer, a feature that is increasingly requested for the absorbent padding requested by the market.

### SUMMARY

The object of the present invention is to provide a method for making a forming pocket for absorbent padding which is free of the drawbacks disclosed above and is in particular simple and cheap to make.

A further object of the present invention is further to provide a method for making a grid-shaped supporting structure in a forming pocket for absorbent padding that enables the shape of the absorbent padding to be made to be altered without the need to replace the production equipment.

Another object of the present invention is additionally to provide a method for making a grid-shaped supporting structure in a forming pocket for absorbent padding that has great production efficiency with reduced machining time for each supporting structure.

An additional object of the present invention is to further provide a forming pocket for absorbent padding that comprises a supporting structure that is simple and cheap to make.

Still another object of the present invention is to further provide a forming pocket for absorbent padding that comprises a supporting structure by means of which it is possible to vary the density of the absorbent padding inside the absorbent padding by means of differentiated sucking zones.

According to the present invention a method is provided for making a forming pocket for absorbent padding according to what has been claimed in the attached claims.

According to the present invention there is further provided a forming pocket for absorbent padding according to what has been claimed in the attached claims.

### BRIEF DESCRIPTION OF THE DRAWINGS

The present invention will now be disclosed with reference to the attached drawings that illustrate some embodiments thereof by way of non-limiting example in which:

FIGS. 1 to 3 illustrate three schematic perspective views of three alternative embodiments of a forming drum for forming absorbent padding for hygienic products, according to the prior art;

FIG. 4 shows a perspective exploded view of a portion of a forming pocket, in which some parts have been removed for the sake of clarity, according to the prior art;

FIG. 5 shows a grid-shaped supporting structure according to the prior art;

FIG. 6 is a perspective top view of a grid-shaped supporting structure according to the invention, in which an external face is visible;

FIG. 7 is a bottom perspective view of the supporting structure of FIG. 6, in which an internal face is visible;

FIG. 8 is an enlarged perspective view of the supporting structure of FIG. 6;

FIG. 9 is an enlargement of FIG. 8;

FIG. 10 is a top front view of the supporting structure of FIG. 6;

FIG. 11 is a front view of a larger side face of the supporting structure of FIG. 6;

FIG. 12 is a bottom front view of the supporting structure of FIG. 6;

FIG. 13 is a front view of a smaller side face of the supporting structure of FIG. 6;

FIG. 14 is a front view of the other smaller side face of the supporting structure of FIG. 6, which is opposite the smaller side face of FIG. 13;

FIG. 15 is a first section view of FIG. 14, along line XV-XV;

FIG. 16 is a second section view of FIG. 14, along line XVI-XVI;

FIG. 17 is a third section view of FIG. 14, along line XVII-XVII;

FIG. 18 is a perspective top view of a different embodiment of the grid-shaped supporting structure according to the invention, in which an external face is visible;

FIG. 19 is a bottom perspective view of the grid-shaped supporting structure of FIG. 18, in which an internal face is visible;

FIG. 20 is an enlargement of a portion of the external face of FIG. 18;

FIG. 21 is an enlargement of a portion of the internal face of FIG. 19;

FIG. 22 is a top front view of the grid-shaped supporting structure of FIG. 18;

FIG. 23 is a bottom front view of the grid-shaped supporting structure of FIG. 18;

FIG. 24 is a section view of FIG. 22, along line XXIV-XXIV;

FIG. 25 is an enlargement of FIG. 24;

FIG. 26 is a section view of FIG. 22, along line XXVI-XXVI.

### DETAILED DESCRIPTION

In this description, identical elements common to the embodiments illustrated are indicated by the same numbering.

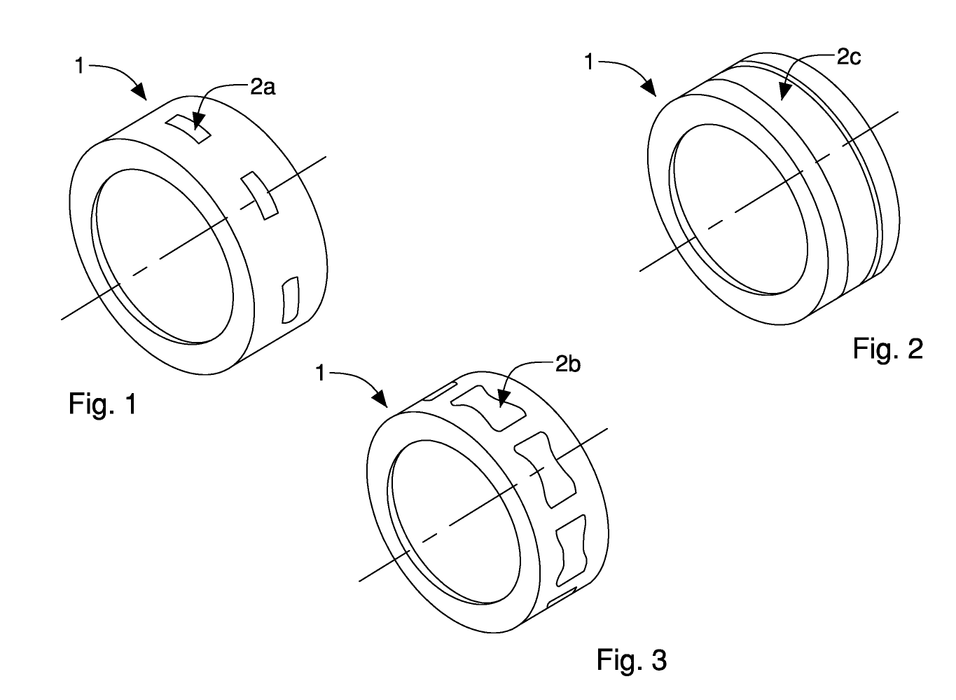

A forming apparatus (which is not shown) for making absorbent padding for hygienic products comprises a forming conveyor of the absorbent padding. A drum forming conveyor has been indicated with 1 in FIGS. 1 to 3 with particular reference to the prior art and is not disclosed again below for the sake of brevity.

The forming conveyor comprises at least one forming pocket (not shown).

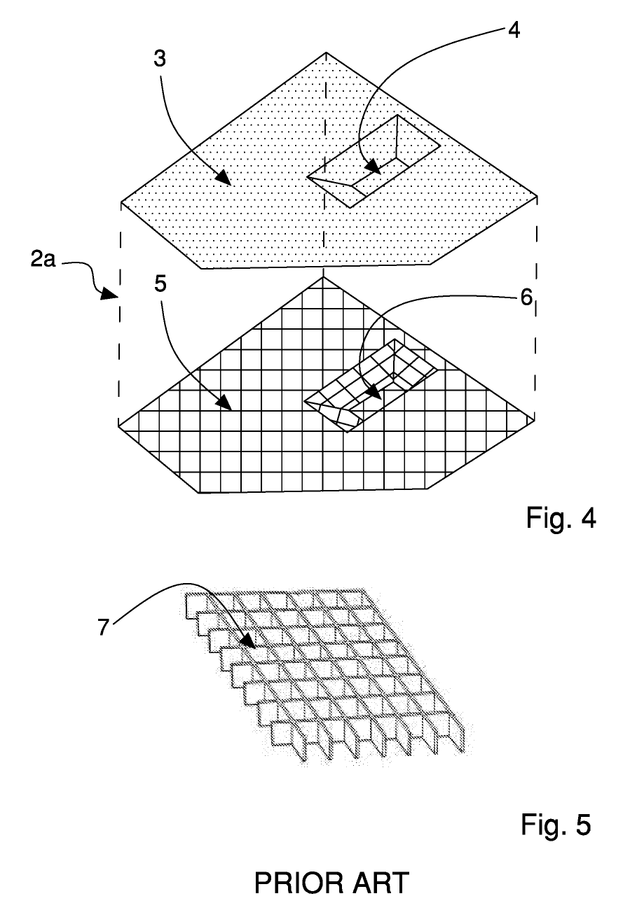

The forming pocket is suitable for receiving particulate material and forming conglomerates from the particulate material to be used as absorbent padding for hygienic products. The forming pocket comprises an external forming substrate which is suitable for receiving the particulate material, which is manufacturable by a metal net or metal sheet, which is provided with openings and has a shape conjugated to the shape of the absorbent padding to be made. The external forming substrate, indicated with 3 in FIG. 4, has already been disclosed in detail with particular reference to the prior art and for the sake of brevity is not disclosed again below.

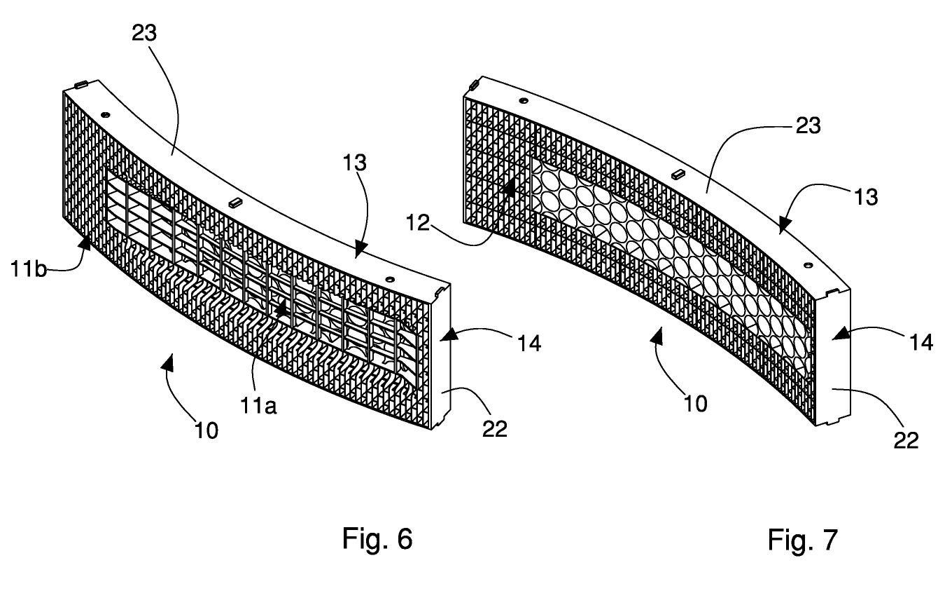

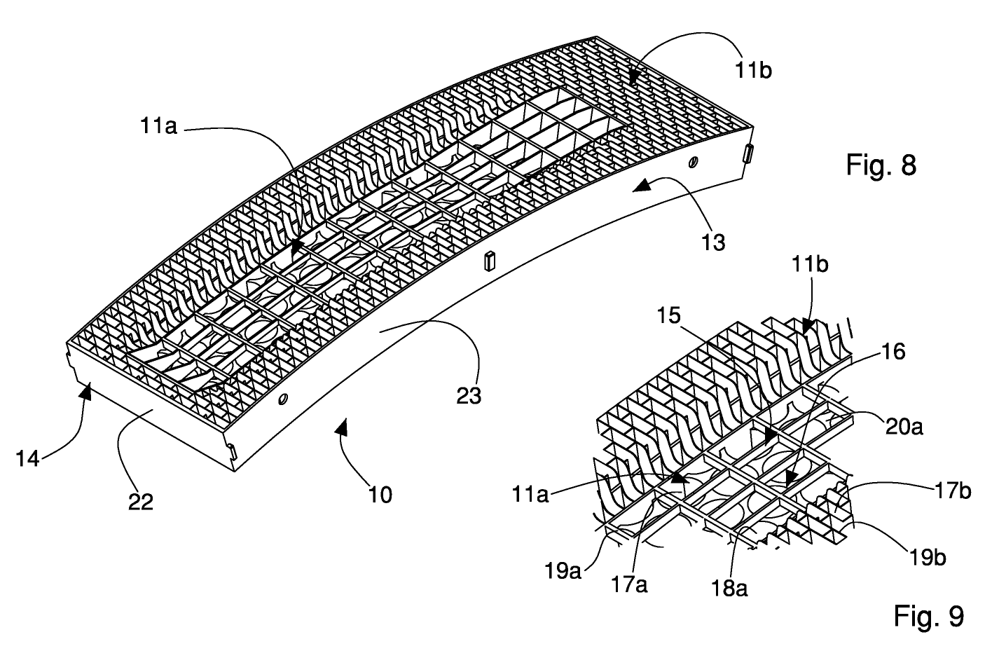

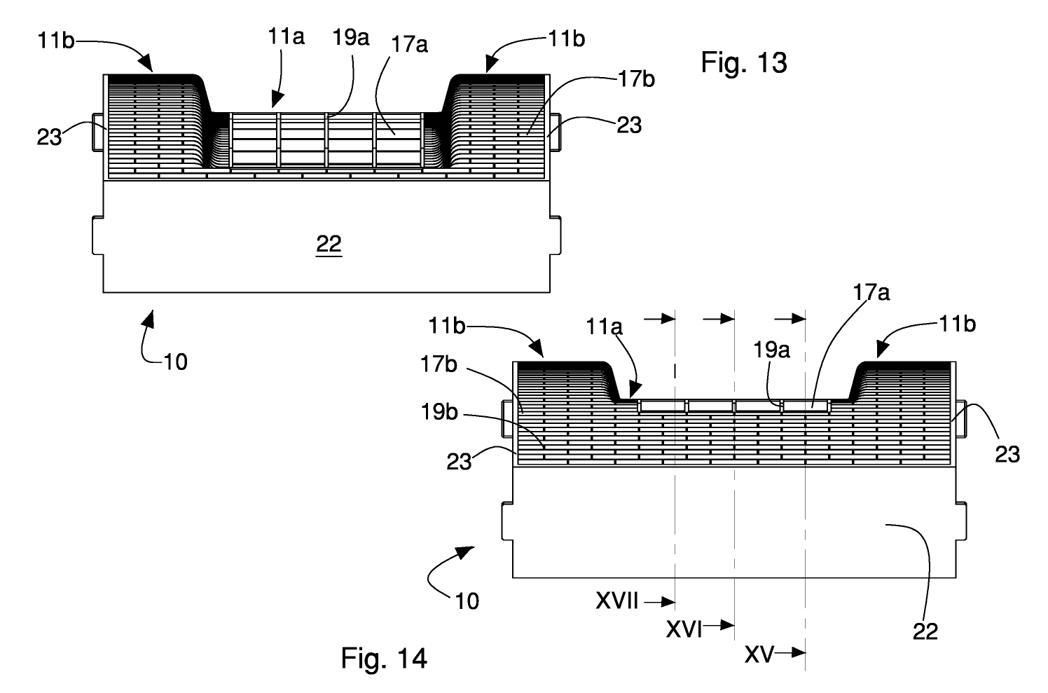

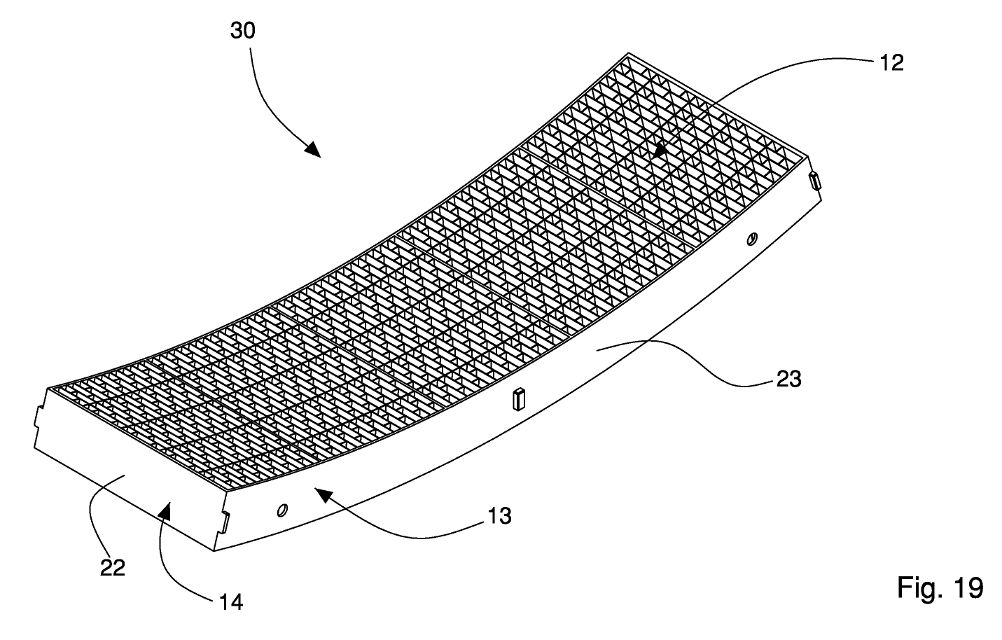

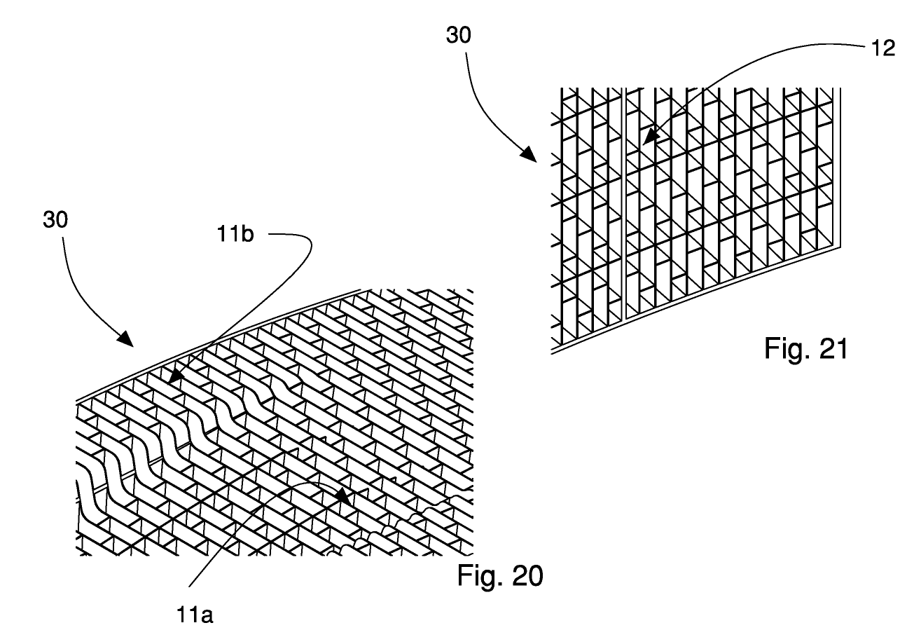

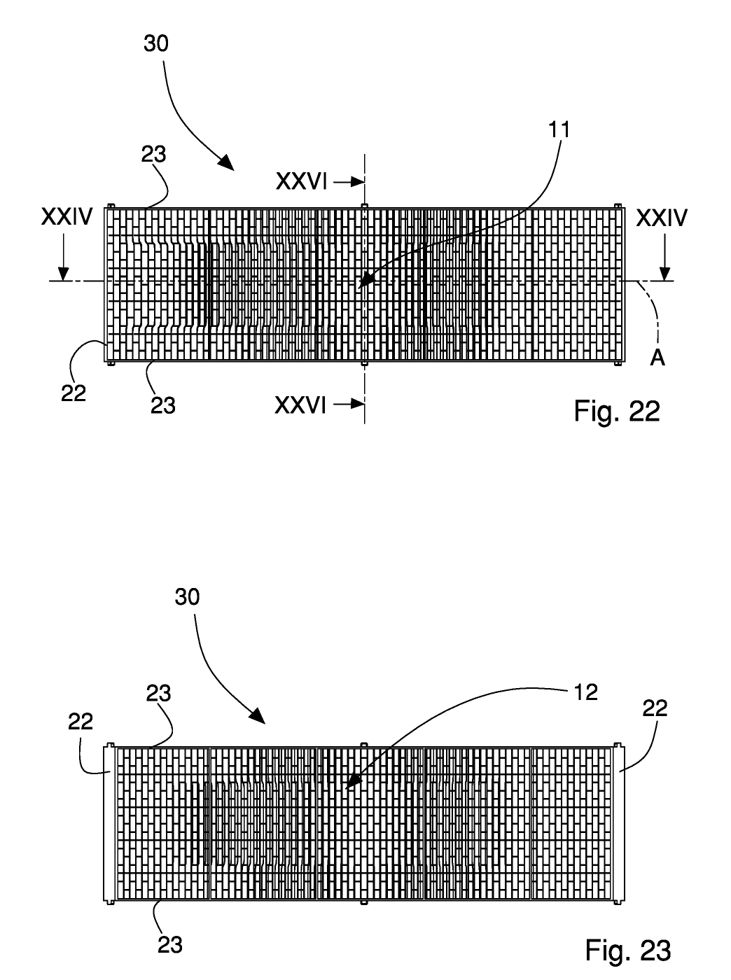

In FIGS. 6 to 26, with 10 a grid-shaped supporting structure is indicated that is provided with openings, which is coupled with the external forming substrate 3 to support the external substrate 3 during suction of the particulate material through the external substrate 3 to form the conglomerate of absorbent material. The external substrate 3 is in particular coupled through superimposing and is fixed to the supporting structure 10.

The supporting structure 10 has a curved external face 11 which is intended for contact with the external substrate and is of a shape conjugated to the shape of the external substrate. The supporting structure additionally has an internal face 12, opposite the external face 11, a pair of greater side faces 13 that are opposite one another and a pair of lesser side faces 14 that are opposite one another, and through openings extending between the external face 11 and the internal face 12 to enable a gas to flow from the external face 11 to the internal face 12 during suction.

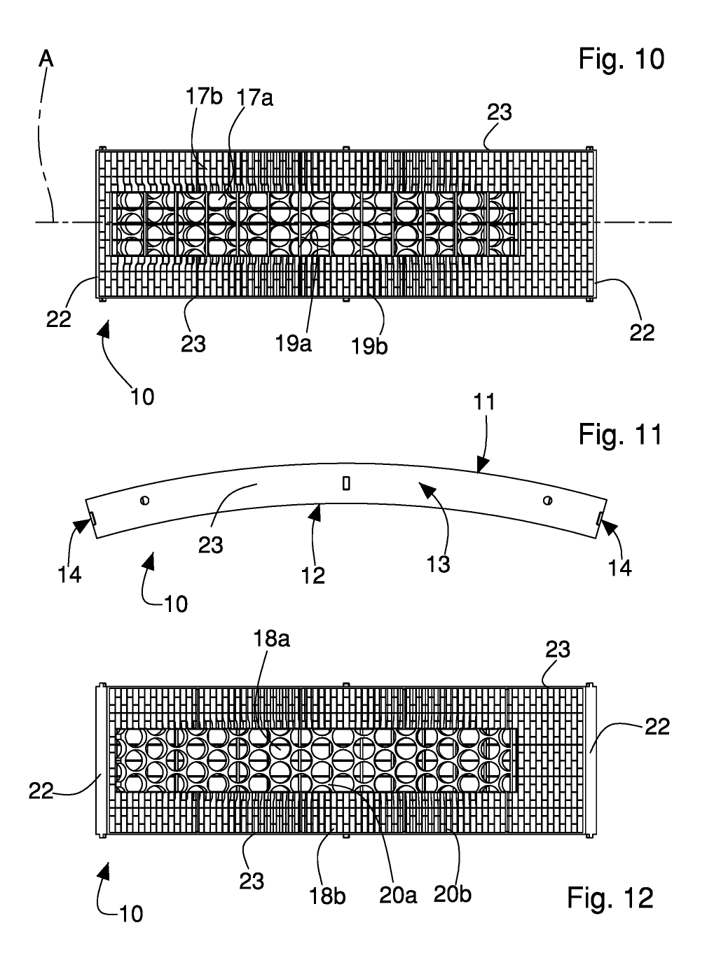

The supporting structure 10 additionally has a longitudinal axis A and a transverse axis B shown in FIG. 10.

The external face 11 has a central zone 11 a provided with a cavity, which extends primarily along the longitudinal axis A and is intended for receiving a corresponding cavity of the external substrate 3, and a curved marginal zone 11b surrounding the central zone 11a, which extends over the remaining part of the external face 11.

The grid supporting structure 10 comprises at least one external layer 15 and an internal layer 16 that are superimposed, having respectively external openings 17 and internal openings 18, which are arranged superimposed so as to define the through openings of the supporting structure 10.

The external openings 17 and the internal openings 18 are bounded by external walls 19 and by internal walls 20.

The external face 11 belongs to the external layer 15 whereas the internal face 12 belongs to the internal layer 16.

According to the present invention, at least one of the external openings 17 has a shape and/or dimension that is different from one of the internal openings 18 on which it is superimposed.

This is possible because the external walls 19 and the internal walls 20 are made by a layer additive manufacturing process, in other words by 3D printing.

The layer additive manufacturing process is selected from the group comprising Selective Laser Sintering-SLS and Selective Laser Melting-SLM, if the material added by layers is selected from the group comprising powder from plastics, metals or ceramics, the metal powder being opportunely selectable from steel, aluminium alloy or titanium alloy powder.

The layer additive manufacturing process is on the other hand selected as Fused Deposition Modelling-FDM if the material added by layers is a filament made of plastics or a metal wire.

The layer additive manufacturing process will be disclosed below in greater detail in this description.

The external layer 15 comprises first external openings 17a between the external openings 17 and the internal layer 16 comprises first internal openings 18a between the internal openings 18, in which the first external openings 17a are superimposed on the first internal openings 18a and in which each opening between the first external openings 17a has a dimension that is greater than one of the first internal openings 18a on which it is superimposed.

The external layer 15 further comprises second external openings 17b between the external openings 17 and the internal layer 16 comprises second internal openings 18b between the internal openings 18, in which the second external openings 17b are superimposed and aligned on the second external openings 18b and have the same dimension as the second internal openings 18b.

It should be noted that the external face 11 of the supporting structure has a central zone 11a provided with a cavity, intended for receiving a corresponding cavity of the external substrate 3 and a curved marginal zone 11b surrounding the central zone 11a that extends over the remaining part of the external face 11.

As is shown in FIGS. 6 to 12, the first external openings 17a and the first internal openings 18a are arranged in the central zone 11a of the supporting structure 10, the second external openings 17b and the second internal openings 18b are arranged in the marginal zone 11b of the supporting structure 10. The dimension of the second external openings 17b and of the second internal openings 18b is less than or the same as the dimension of the first internal openings 17a.

In other words, at the central zone 11a, the external layer 15 is of the net type and has a plurality of first external openings 17a with a rectangular section whereas the internal layer 16 is of the honeycomb type and has a plurality of first internal openings 18a with a circular section.

The shape of the first external openings 17a is thus different from the shape of the first internal openings 18a on which the first external openings 17a are superimposed. It should be noted in addition that also the dimension of the first external openings 17a and of the first internal openings 18a is different, inasmuch as the first external openings 17a are larger than the first internal openings 18a, to convey in a controlled manner the sucking air from the external face 11 to the internal face 12 of the supporting structure 10.

On the other hand, at the marginal zone 11b, the shape of the second external openings 17b corresponds to the shape of the second internal openings 18b inasmuch as, for example, it is not required to make absorbent padding with zones with differentiated density.

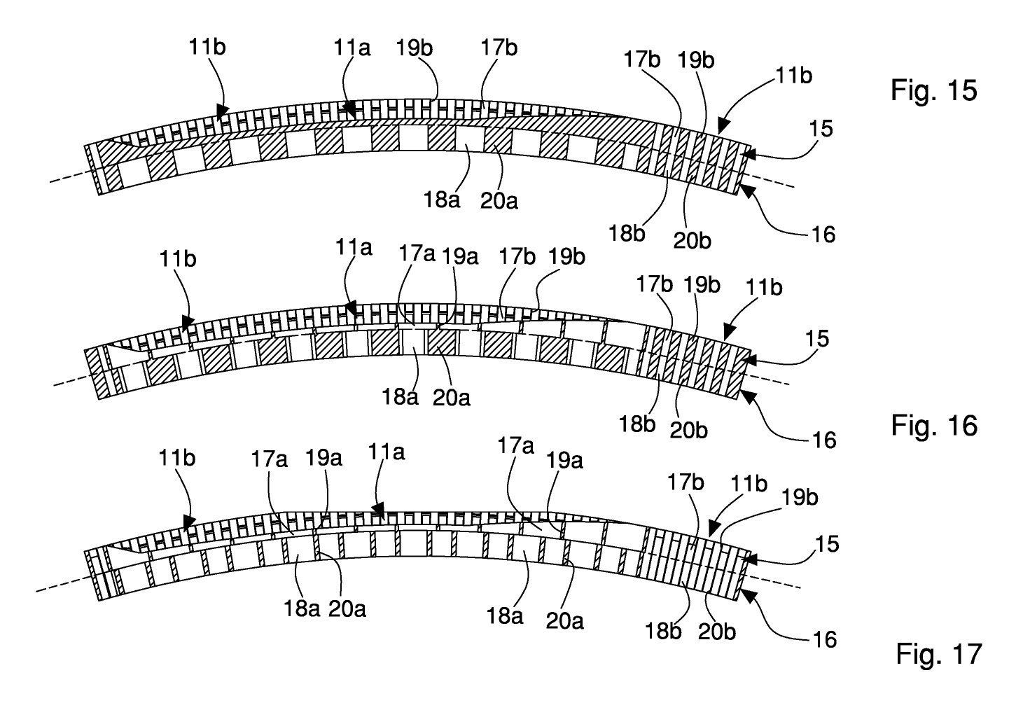

The different arrangement, shape and dimension of the first external openings 17a and of the first internal openings 18a from the second external openings 17b and from the second internal openings 18b is clearly shown in FIGS. 15 to 17, which show different longitudinal sections of the supporting structure 10.

In FIG. 17 it can for example be remarked that the first external openings 17a are superimposed on the first internal openings 18a and are of a greater dimension than the latter. In view of the different dimension between the first external openings 17a and the first internal openings 18a, the external walls 19 are staggered with respect to the internal walls 20.

The external walls 19 are on the other hand aligned on the internal walls 20 at the marginal zone 11b and make single walls that extend without interruption from the external face 11 to the internal face 12 of the supporting structure 10.

According to the present invention, it is added that at least one of the external walls 19 has a different thickness from one of the internal walls 20 on which it is superimposed to bound respective external openings 17 superimposed on respective internal openings 18 of different shape and/or dimensions.

The external layer 15 comprises first external walls 19a between the external walls 19 and the internal layer 16 comprises first internal walls 20a between the internal walls 20, in which the first external walls 19a are superimposed on the first internal walls 20a, the first internal walls 20a have greater thickness than the first external walls 19a on which they are superimposed, so that the first internal walls 20a are reinforcing walls.

In detail, the internal layer 16 further comprises second internal walls 20b, in which respectively the first external walls 19a have a constant first thickness, the first internal walls 20a have a constant second thickness and the second internal walls 20b have a constant third thickness, and in which the second thickness is greater than both the first thickness and the third thickness.

In other words, the internal layer 16 is made with first internal walls 20a and second internal walls 20b which are of different thickness from one another and the first internal walls 20a have a greater thickness than both the first external walls 19a and the second internal walls 20b to make reinforcing walls in specific portions of the supporting structure 10.

The external layer 15 further comprises second external walls 19b superimposed on the second internal walls 20b, the thickness of the second external walls 19b being the same as the thickness of the second internal walls 20b. If the thickness of the first external walls 19a and of the second external walls 19b is the same, the external layer 15 has walls 19 of uniform thickness.

As the supporting structure 10 is made by a layer additive manufacturing process, i.e. by 3D printing, the first internal walls 20a and second internal walls 20b of differentiated thickness are made simultaneously.

At the central zone 11a of the supporting structure 10, it can be noted (FIG. 16) that first internal walls 20a are present the thickness of which is greater than the thickness of the first external walls 19a, and second internal walls 20b are further present, the thickness of which is less than the thickness of the first internal walls 20a (FIG. 17).

The supporting structure 10 further comprises a stabilisation frame comprising a pair of opposite lesser laminar elements 22 suitable for defining the lesser side faces of the supporting structure 10 and a pair of opposite greater laminar elements 23, suitable for defining the greater side faces of the supporting structure 10.

The grid-shaped supporting structure 10 and stabilisation frame are made simultaneously, in particular the stabilisation frame is made simultaneously with the external layer 5 or with the internal layer 16 inasmuch as the stabilisation frame and the external layer 15 or the internal layer 16 are made by an additive manufacturing process.

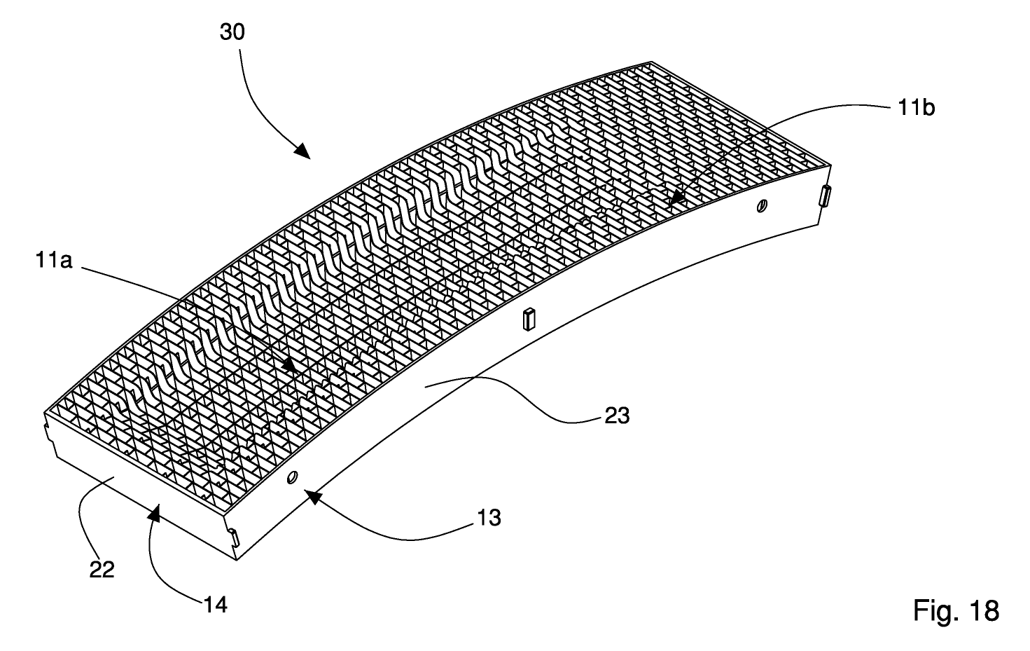

According to a different embodiment shown in FIGS. 18 to 26, with 30 a supporting structure is indicated in which the first external openings 17a and the first internal openings 18a are not localised in the central zone 11a but are distributed in the supporting structure 10.

In the supporting structure 30 only the arrangement is different of the external openings 17 and of the respective internal openings 18, which have a shape and/or dimension that is different from the supporting structure 10, but everything said previously about the supporting structure 10 still remains valid.

The external layer 15 in fact has first external walls 19a and second external walls 19b that bound respective external openings 17 superimposed on respective internal openings 18 of different shape and/or dimensions.

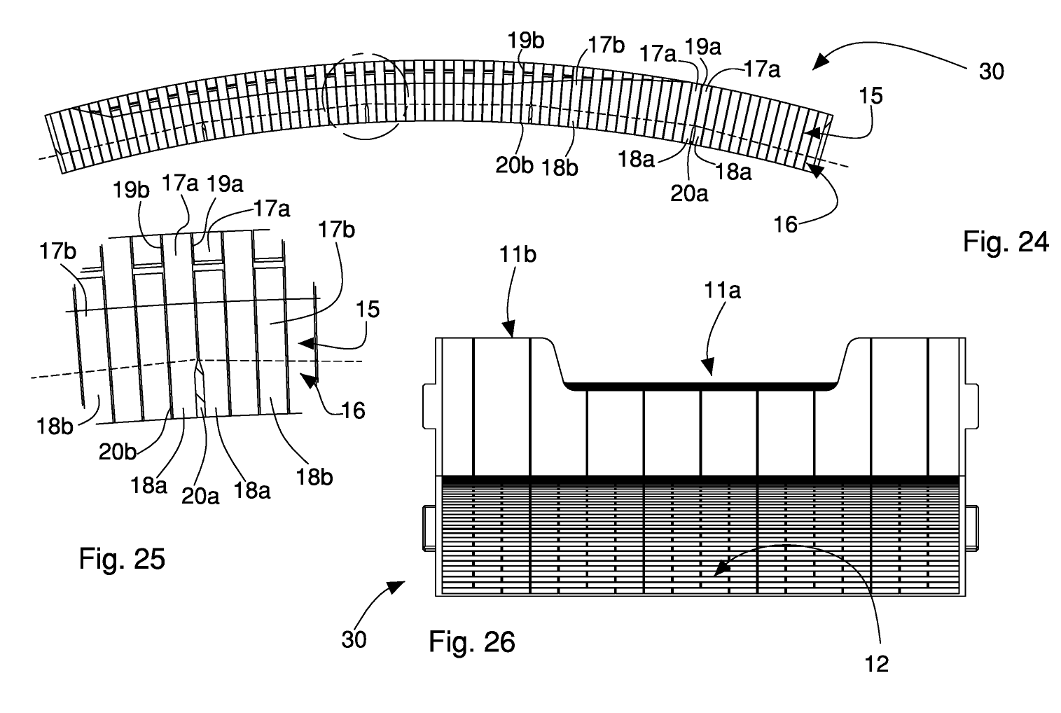

As is clear in particular in FIGS. 23 and 24, the internal layer 16 is made with first internal walls 20a and second internal walls 20b, in which the first internal walls 20 make reinforcing walls the thickness of which is greater than both the thickness of the second external walls 20b and the thickness of the first external walls 19a and the arrangement of the first internal walls 20a is such that they are distributed uniformly between the second internal walls 20b.

In detail, the reinforcing first internal walls 20a are radially equidistant between the second internal walls 20b and define part of the internal face 12 of the supporting structure 10.

In the supporting structure 30 shown in FIGS. 18 to 26, the external walls 19 are thus consecutive and adjacent to the internal walls 20, and the first external openings 17a of greater dimension than the corresponding first internal openings 18a on which they are superimposed are distributed uniformly between the second external openings 17b and the second internal openings 18b having the same dimension. The different dimension between the first external openings 17a and first internal openings 18a is thus due to the different thickness between the first external walls 19a and the first internal walls 20b.

Further, in a localised and distributed manner on the supporting structure 30, it is thus possible to have reinforcing walls of the reinforcing structure 30.

According to a version that is not shown, the forming pocket can comprise a supporting structure in which the central zone 11 a is configured as in FIGS. 6 to 17 and has first external openings 17a of a larger dimension than first internal openings 18a on which the first external openings 17a are superimposed, and first external walls 19a of a lesser thickness than first internal walls 20a, and further a marginal zone 11b in which the first external openings 17a of a larger dimension than first internal openings 18a are distributed in the marginal zone, to have reinforcing first internal walls 20a that are distributed in the marginal zone.

In other words, it is possible to have different embodiments of the supporting structure of the present invention, which are not shown, by arranging differently in the supporting structure the external openings 17 of the external layer 15 and the internal openings 18 of the internal layer 16 that are of different shape and/or dimension from one another.

Owing in fact to manufacture of the walls of the supporting structure by a layer additive manufacturing process, i.e. by 3D printing, it is possible to make a very complex grid-shaped supporting structure, comprising at least one external layer 15 and an internal layer 16 in which each layer has openings of different shape and/or dimensions and in which, further, walls bounding the external layer 15 or the internal layer 16 can be unaligned, which is otherwise not achievable with prior art spark discharge machining processes.

It is added that if a forming apparatus is provided for making an absorbent padding for hygienic products that comprises a forming conveyor of the absorbent padding comprising at least one forming pocket according to what has been disclosed previously and the forming conveyor is a forming drum, the internal face 12 of the supporting structure is curved, as illustrated in FIGS. 6 to 26, inasmuch as it is intended for contact with the forming drum, and in particular has a shape conjugated to an external face of the forming drum.

A method is further disclosed for making a grid-shaped supporting structure 10 which is couplable with an external substrate 3 of a forming pocket, in which the external substrate 3 is provided with openings and has a shape conjugated to the shape of the absorbent padding to be made.

In order to make a forming pocket suitable for receiving particulate material and forming conglomerates from said particulate material to be used as absorbent padding for hygienic products, a method is proposed that comprises:
providing an external forming substrate 3, which is suitable for receiving the particulate material, which is of a shape conjugated to the shape of the absorbent padding to be made and further providing openings in the external forming substrate 3;
providing a grid-shaped supporting structure 10, which is couplable with the external substrate 3 to support the external substrate 3 during suction of the particulate material through the external substrate 3 and providing in the grid-shaped supporting structure 10 a curved external face 11 intended for contact with the external substrate 3 and of a shape conjugated to the shape of the external substrate 3, an internal face 12, opposite the external face 11, a pair of greater side faces 13 that are opposite one another and a pair of lesser side faces 14 that are opposite one another, and through openings extending between the external face 11 and the internal face 12 to enable a gas to flow from the external face 11 to the internal face 12 during suction of the particulate material.

The method further comprises:
making at least one external layer 15 of the supporting structure 10 and at least one internal layer 16 of the supporting structure 10 that are superimposed and have respectively external openings 17a, 17b and internal openings 18a, 18b bounded by external walls 19a, 19b and by internal walls 20a, 20b;
arranging the external openings 17a, 17b and the internal openings 18a, 18b superimposed so as to define the through openings;
making at least one of the external openings 17a, 17b of a different shape and/or dimension from one of the internal openings 18a, 18b on which it is superimposed;
and in which the method further comprises making the external walls 19a, 19b and the internal walls 20a, 20b by a layer additive manufacturing process, i.e. by 3D printing (layer additive manufacturing process).

The layer additive manufacturing process, i.e. the 3D printing process, enables walls of the desired dimension and of the desired shape to be made simply and cheaply that are arranged in any position of the supporting structure 10 without the need to use dedicated labour, equipment and dedicated manufacturing processes.

Manufacturing times are reduced and tooling costs are thus eliminated.

Further, the 3D printing process enables external openings 17 and internal openings 18 to be made that are superimposed and are of dimensions and/or of shapes that are different from one another, arranging suitably the respective external walls 19 and internal walls 20, which would be difficult to make with traditional productive processes.

Additive manufacturing or the additive process or the layer additive manufacturing process is a known process of joining materials to manufacture three-dimensional objects from computerised 3D models, usually one layer above the other.

Different 3D printing technologies exist and the main differences between them relate to the manner in which the layers are printed, which depends also on the material used to make the object of interest.

If a 3D printing is used by means of the Selective Laser Sintering-SLS and Selective Laser Melting-SLM method, a laser source is used to transform (or sinter) by high-temperature heat treatment a powder material into an indivisible material, creating by layers a three-dimensional object. The SLS or SLM method makes the object by layers by spreading a very thin layer of powder on a work platform and melting the powder by means of the laser on the basis of the geometry established for each layer. The material can be in this case selected as a plastics, metal or ceramic material and in detail the metal powders are selectable from steel, aluminium alloy or titanium alloy powders.

On the other hand, in the case of 3D printing by Fused Deposition Modelling-FDM, a material is dispensed melted by layers by an extrusion nozzle, which is movable both horizontally and vertically and is controlled by a numerically controlled system. The material is supplied to the extrusion nozzle as a thread, of plastics or of metal material, and is dissolved at the nozzle before deposition.

Owing to the possibility of making the external layer 15 and the internal layer 16 by a layer additive manufacturing process, first external openings 17a are made between the external openings 17 and first internal openings 18a between the internal openings 18, in which the first external openings 17a are superimposed on the first internal openings 18a and in which each opening between the first external openings 17a has a dimension that is greater than one of the first internal openings 18a on which it is superimposed.

By further making second external openings 17b between the external openings 17 and second internal openings 18b between the internal openings 18, in which the second external openings 17b are superimposed and aligned on the second internal openings 18b and have the same dimension as the second internal openings 18b, it is possible to create differentiated sucking zones of the supporting structure.

Nevertheless, to create first external openings 17a of greater dimension than the openings first internal openings 18a to which they are subjected, it is also possible to make at least one of the external walls of a different thickness from one of the internal walls on which it is superimposed, thus limiting external openings 17 superimposed on respective internal openings 18 of different shape and/or dimensions.

The position and the thickness of the external walls 19 and of the internal walls 20 determines the shape and/or the dimension of the external openings 17 and of the internal openings 18.

First external walls 19a and first internal walls 20a are created, in which the first external walls 19a are superimposed on the first internal walls 20a and the first internal walls 20a have greater thickness than the first external walls 19a so that the first internal walls 20a are reinforcing walls of the supporting structure 10.

It can be in particular economical to make second internal walls 20b between the internal walls 20, in which respectively the first external walls 19a have a constant first thickness, the first internal walls 20a have a constant second thickness and the second internal walls 20b have a constant third thickness, in which the second thickness is greater than both the first thickness and the third thickness and in particular the first thickness is equal to the third thickness.

Clearly, the internal walls 20 of the internal layer 16, both the first internal walls 20a and the second internal walls 20b, are made simultaneously simply and cheaply, according to what the geometry is of the desired supporting structure, owing to 3D printing.

In order to make the supporting structure 10 sturdier, making a stabilisation frame of the supporting structure 10 is provided for that comprises a pair of opposite lesser laminar elements 22 suitable for defining the lesser side faces 14 of the supporting structure 10 and a pair of opposite greater laminar elements 23, suitable for defining the greater side faces 13 of the supporting structure 10. The stabilisation frame is made, by layers, simultaneously to the external walls 19 of the external layer 15 or to the internal walls 20 of the internal layer 16.

In fact, owing to the manufacture of the walls of the supporting structure by a layer additive manufacturing process, i.e. by 3D printing, it is possible to make a very complex grid-shaped supporting structure comprising at least one external layer 15 and an internal layer 16 in which each layer has openings of different shape and/or dimensions and in which, further, the walls bounding the external layer 15 or the internal layer 16 can be unaligned.

This complex grid-shaped supporting structure 10, made by 3D printing, enables differentiated sucking zones of particulate material to be created that permit advantageous use thereof in a forming pocket of a forming apparatus for forming absorbent padding. The same supporting structure could not be manufacturable with prior-art spark discharge machining processes and if it were, would have such high costs as to make the industrial applicability thereof impossible.

1. A method for making a forming pocket suitable for receiving particulate material and forming conglomerates from said particulate material to be used as absorbent padding for hygienic products, in which the method comprises:
providing an external forming substrate, which is suitable for receiving the particulate material, of a shape conjugated to the form of the absorbent padding to be made and providing openings in said external forming substrate;
providing a grid-shaped supporting structure, which is couplable with the external substrate to support said external substrate during suction of the particulate material through the external substrate, and providing in the grid-shaped supporting structure a curved external face, intended for contact with the external substrate and of a shape conjugated to the shape of the external substrate an internal face, opposite the external face, a pair of greater side faces that are opposite one another and a pair of lesser side faces that are opposite one another, and through openings extending between the external face and the internal face to enable a gas to flow from the external face to the internal face during said suction; in which the method further includes:
making at least one external layer of the supporting structure and at least one internal layer of the supporting structure that are superimposed and have respectively external openings and internal openings bounded by external walls and by internal walls;
arranging the external openings and the internal openings superimposed so as to define said through openings;
making at least one of the external openings of a different shape and/or dimension from one of the internal openings on which it is superimposed; and in which the method further includes making the external walls and the internal walls by a layer additive manufacturing process, i.e. by 3D printing.

2. The method according to claim 1, and including making first external openings between the external openings and first internal openings between the internal openings, in which the first external openings are superimposed on the first internal openings and in which each opening between the first external openings has a dimension that is greater than one of the first internal openings on which it is superimposed.

3. The method according to claim 2, and further including making second external openings between the external openings and second internal openings between the internal openings, in which the second external openings are superimposed and aligned on the second internal openings and have the same dimension as the second internal openings.

4. The method according to claim 1, and including making at least one of the external walls of a different thickness from one of the internal walls on which it is superimposed to bound respective external openings superimposed on respective internal openings of different shape and/or dimensions.

5. The method according to claim 4, and including first external walls between the external walls and first internal walls between the internal walls, in which the first external walls are superimposed on the first internal walls and the first internal walls have greater thickness than the first external walls so that the first internal walls are reinforcing walls.

6. The method according to claim 5, and further including making second internal walls between the internal walls, in which respectively the first external walls have a constant first thickness, the first internal walls have a constant second thickness and the second internal walls have a constant third thickness, in which the second thickness is greater than both the first thickness and the third thickness, in particular the first thickness being the same as the third thickness, and making the first internal walls simultaneously to the second internal walls.

7. The method according to claim 1, and further including making a stabilisation frame of the supporting structure including a pair of opposite lesser laminar elements suitable for defining the lesser side faces of the supporting structure and a pair of opposite greater laminar elements, suitable for defining the greater side faces of the supporting structure, and in which the method further includes making the stabilisation frame simultaneously to the external walls or to the internal walls.

8. The method according to claim 1, and further including selecting the layer additive manufacturing process, i.e. the 3D printing, in the group including Selective Laser Sintering-SLS and Selective Laser Melting-SLM if the material added by layers is selected from the group including powder from plastics, metals or ceramics, the metal powder being opportunely selectable from steel, aluminium alloy or titanium alloy powder; or selecting the layer additive manufacturing process method like Fused deposition modelling-FDM if the material added by layers is a filament made of plastics or a metal wire.

9. A forming pocket, suitable for receiving particulate material and forming conglomerates from said particulate to be used as absorbent padding for hygienic products, in which the forming pocket comprises:
an external forming substrate which is suitable for receiving the particulate material, which is provided with openings and has a shape conjugated to the form of the absorbent padding to be made;
a grid-shaped supporting structure, which is couplable with the external substrates to support said external substrates during suction of the particulate material through the external substrates and includes a curved external face, intended for contact with the external substrate and of a shape conjugated to the shape of the external substrate, an internal face, opposite the external face, a pair of greater side faces that are opposite one another and a pair of lesser side faces that are opposite one another, and through openings extending between the external face and the internal face to enable a gas to flow from the external face to the internal face during suction;
wherein the grid-shaped supporting structure includes at least one external layer and one internal layer that are superimposed and have respectively external openings and internal openings, respectively bounded by external walls and by internal walls, which are arranged superimposed so as to define said through openings; and wherein at least one of the external openings has a shape and/or dimension that is different from one of the internal openings on which it is superimposed, the external walls and the internal being made by a layer additive manufacturing process, i.e. by 3D printing.

10. The forming pocket according to claim 9, wherein the external layer includes first external openings between the external openings and the internal layer includes first internal openings between the internal openings in which the first external openings are superimposed on the first internal openings and in which each opening between the first external openings has a dimension that is greater than one of the first internal openings on which it is superimposed.

11. The forming pocket according to claim 10, wherein the external layer includes second external openings between the external openings and the internal layer includes second internal openings between the internal openings, in which the second external openings are superimposed and aligned on the second internal openings and have the same dimension as the second internal openings.

12. The forming pocket according to claim 11, wherein the external face has a central zone, provided with a cavity, intended for receiving a corresponding cavity of the external substrate and a curved marginal zone surrounding the central zone that extends over the remaining part of the external face, the first external openings and the first internal openings being arranged in the central zone of the supporting structure, the second external openings and the second internal openings being arranged in the marginal zone of the supporting structure.

13. The forming pocket according to claim 12, wherein the dimension of the second external openings and of the second internal openings is less than or the same as the dimension of the first internal openings.

14. The forming pocket according to claim 9, wherein at least one of the external walls has a different thickness from one of the internal walls on which it is superimposed to bound respective external openings superimposed on respective internal openings of different shape and/or dimensions.

15. The forming pocket, according to claim 14, wherein the external layer includes first external walls between the external walls and the internal layer includes first internal walls between the internal walls, in which the first external walls are superimposed on the first internal walls, the first internal walls having a greater thickness than the first external walls so that the first internal walls are reinforcing walls.

16. The forming pocket according to claim 15, wherein the internal layer includes second internal walls, in which respectively the first external walls have a constant first thickness, the first internal walls have a constant second thickness and the second internal walls have a constant third thickness, and wherein the second thickness is greater than both the first thickness and the third thickness, in particular the first thickness being the same as the third thickness, and further wherein the first internal walls and the second internal walls are made simultaneously.

17. The forming pocket according to claim 16, wherein first internal walls are distributed uniformly between the second internal walls in particular are spaced radially equidistantly, and define a portion of the internal face of the supporting structure.

18. The forming pocket according to claim 14, wherein the external walls are consecutive and adjacent to the internal walls.

19. The forming pocket according to claim 9, wherein the supporting structure further includes a stabilisation frame including a pair of opposite lesser laminar elements suitable for defining the lesser side faces of the supporting structure and a pair of opposite greater laminar elements, suitable for defining the greater side faces of the supporting structure, wherein the grid-shaped supporting structure e la stabilisation frame are made simultaneously by a layer additive manufacturing process, i.e. by 3D printing.

20. A forming apparatus for making an absorbent padding for hygienic products, including a forming conveyor of the absorbent padding including at least one forming pocket according to claim 9, wherein the forming conveyor is a forming drum and the internal face of the supporting structure is curved, being intended for contact with the forming drum, said internal face being of a shape conjugated to an external face of the forming drum.
